(19) United States
(12) Patent Application Publication
(10) Pub. No.: US 2015/0374741 A1
(43) Pub. Date: Dec. 31, 2015

(54) METHODS FOR MAINTAINING POPULATION OF THERAPEUTIC CELLS IN TREATMENT SITE OF SUBJECT IN NEED OF CELL THERAPY

(71) Applicants: DCB-USA LLC, Wilmington, DE (US); NATIONAL CHENG KUNG UNIVERSITY, Tainan City (TW)

(72) Inventors: Patrick Ching-Ho HSIEH, Tainan City (TW); Chia-Yun LAI, Hsinchu County (TW)

(21) Appl. No.: 14/767,774

(22) PCT Filed: Feb. 14, 2014
(86) PCT No.: PCT/US14/16477

§ 371 Date: Aug. 13, 2015

Related U.S. Application Data
(60) Provisional application No. 61/765,075, filed on Feb. 15, 2013.

(51) Int. Cl. A61K 31/728; A61K 35/28; A61K 35/12

(52) U.S. Cl. CPC A61K 31/728; A61K 35/12; A61K 35/28

(57) ABSTRACT

Disclosed herein are methods for maintaining a population of therapeutic cells administered to a treatment site in a subject in need of cell therapy for a period of time. The methods include administering a therapeutically-effective amount of therapeutic cells and an effective amount of a biodegradable material to the treatment site. The biodegradable material includes a hyaluronan compound and has an in vivo degradation profile similar to that of a hyaluronic acid having a molecular weight of 20 kDa to 2,000 kDa.

### CROSS-REFERENCE TO RELATED APPLICATION

This application claims the benefit of U.S. Provisional Application No. 61/765,175, filed 15 Feb. 2013, the entirety of which is incorporated herein by reference.

### BACKGROUND OF THE INVENTION

1. Field of the Invention

The present disclosure relates to cell therapies. More particularly, the disclosed invention relates to improving the therapeutic efficacy of cell therapies using at least one hyaluronan compound.

2. Description of Related Art

According to the definition by the U.S. Food and Drug Administration (FDA), somatic cell therapy (or cell therapy) is the prevention, treatment, cure, diagnosis, or mitigation of diseases or injuries in humans by the administration of autologous, allogeneic or xenogeneic cells that have been manipulated or altered ex vivo. Generally, said manipulation and alteration include the propagation, expansion, selection, and/or pharmacological treatment of the cells.

The goal of cell therapy is to repair, replace or restore damaged tissues or organs. Cell therapy may provide extensive applications in modern medicine. For example, in Nov. 10, 2011, the U.S. FDA granted marketing approval to the New York Blood Center's allogeneic cord-blood product, HEMACORD, the first FDA-licensed hematopoietic progenitor cell therapy. HEMACORD is indicated for hematopoietic progenitor cell (HPC) transplantation procedures in patients with inherited, acquired, or myeloablative-treatment-related diseases that affect the hematopoietic system. Once the HPCs are infused into patients, the cells migrate to the bone marrow where they divide and mature. When the mature cells move into the bloodstream they can partially or fully restore the number and function of many blood cells, including immune function.

There are many factors affecting the therapeutic efficacy of the cell therapy, such as the pre-transplantation preparation of the cells, the transplantation procedure and the in vivo system receiving the cells. Regarding the last aspect, certain obstacles faced by cell therapies include unsatisfactory cell retention at the target site, difficulty in tracing the spatial distribution of transplanted cells, and poor cell survival in adverse microenvironments. These problems may lead to poor therapeutic efficacy of cell therapies.

The related art has attempted to address the above-identified issues by using biodegradable materials, with the goal of controlling the timespan of cell retention in vivo. Ideally, the in vivo duration of the biodegradable material should be sufficiently long for the cells to adhere to the surrounding microenvironment but not so long that the biodegradable material may result in undesirable responses. Better therapeutic outcomes may thus be reached once the critical time point for cell adhesion and function is known.

Unfortunately, most prior art studies involve in vitro assessments of material degradation, and cannot properly mimic the intrinsic complexity of the in vivo systems. For example, changes in the biodegradable material integrity and mass in vivo may affect the degradation profile of the biodegradable material, endowing the material with the distinct capabilities to retain transplanted cells in the target site. Accordingly, determination of the cell delivery capacity of the biodegradable material cannot be adequately assessed solely by in vitro experimentation.

In view of the foregoing, there exists a need in the art for providing developing an alternative strategy that minimizes cell loss in vivo so as to improve the therapeutic efficacy of cell therapy.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to a method for maintaining a population of therapeutic cells administered to a treatment site in a subject for a period of time. In another aspect, the present disclosure is directed to a method for treating a subject in need of cell therapy.

According to one embodiment of the present disclosure, the method comprises the steps of, administering a therapeutically-effective amount of therapeutic cells to the treatment site; and administering an effective amount of a biodegradable material comprising a hyaluronan compound to the treatment site. The biodegradable material has an in vivo degradation profile similar to that of a hyaluronic acid having a molecular weight of 20 kDa to 2,000 kDa.

According to various embodiments of the present disclosure, the therapeutic cells may be stem cells, induced pluripotent cells, functionally differentiated cells, recombinant cells, or a combination thereof.

Examples of stem cells includes but are not limited to, embryonic stem cells, hematopoietic stem cells, vascular stem cells, neural stem cells, mesenchymal stem cells, cardiac stem cells, adipose stem cells, muscular stem cells, dental stem cells, skeletal stem cells, cartilage stem cells, periosteal stem cells, mammary stem cells, uterus stem cells, endothelial stem cells, skin stem cells, placental stem cells, umbilical cord blood stem cells, yolk sac stem cells, and amniotic fluid stem cells. Exemplary functionally differentiated cells include fibroblasts, chondrocytes, osteoblasts, osteocytes, adipocytes, epithelial cells, keratinocytes, retinal cells, dental cells, renal cells, pancreatic islet cells, hepatocytes, neuronal cells, immune cells, muscle cells, and blood cells.

According to certain embodiments of the present disclosure, the hyaluronan compound is any of the following compounds: hyaluronic acid, partial or total esters of hyaluronic acid, adipic dihydrazide-modified hyaluronan, amides of hyaluronan, crosslinked hyaluronic acid, hemiesters of succinic acid, heavy metal salts of hyaluronic acid, sulphated hyaluronic acid, N-sulphated hyaluronic acid, amine-modified hyaluronic acid, diamine-modified hyaluronic acid, and a composite of hyaluronic acid and silk.

In certain embodiment, the in vivo degradation profile of the biodegradable material is similar to that of a hyaluronic acid having a molecular weight of 50 kDa to 1,600 kDa; preferably, 200 kDa to 800 kDa.

According to embodiments of the present disclosure, the population of therapeutic cells at the treatment site is maintained for at least 7 days; preferably at least 14 days; and more preferably, at least 28 days.

In certain embodiments of the present disclosure, the biodegradable material has an in vivo half-life of 4 hours to 28 days; alternatively, the in vivo half-life of the biodegradable material is 8 hours to 7 days, 12 hours to 5 days or 1-3 days after the administration.

Optionally, in some embodiments, the biodegradable material further comprises at least one biopolymer; examples of which include, but are not limited to, collagen, gelatin, alginate, chitosan, fibronectin, and fibrin glue.

According to embodiments of the present disclosure, the population of the therapeutic cells at the treatment site, at 3-10 days after the administration, is substantially the same as or greater than the population of the therapeutic cells initially administered.

In various embodiments, the therapeutic cells are administered prior to, concurrently with, or after the administration of the biodegradable material. In the case of concurrent administration, the therapeutic cells and the biodegradable material may be formulated as a single composition or in separate compositions.

According to embodiments of the present disclosure, the therapeutic cells and the biodegradable material are administered by direct application, catheter-assisted delivery, endoscope-assisted delivery, robotic-assisted delivery, device-assisted delivery, or imaging device-guided delivery, respectively or concomitantly.

In certain embodiments, the therapeutically-effective amount of the therapeutic cells is 1×10⁴ to 1×10⁸ cells/kg body weight of the subject. In these cases, or in other embodiments, the effective amount of the hyaluronan compound is 0.01 to 10 mg/kg body weight of the subject.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 and FIG. 2 demonstrate the linear relationships determined between the fluorescent intensity of Alexa Fluor 700 streptavidin (SA) and Ds-Red expressing human mesenchymal stem cells (hMSCs); R=0.99 for both the streptavidin (FIG. 1) and hMSCs (FIG. 2).

FIG. 3 provides images collected by in vivo imaging system (IVIS) using the corresponding filter sets for the Alexa Fluor 700 streptavidin (SA) and Ds-Red expressing hMSCs; the upper two IVIS images were collected before mixing SA and hMSCs and the lower two images were collected after mixing. Although not visible in these figures, the fluorescent signals were yellow to red and regions of interest (ROls) were blue line-enclosed. 570/620: the excitation/emission filter set for the hMSCs. 675/720: the filter set for the Alexa Fluor 700 SA.

FIG. 4 provides bar graphs demonstrating the statistical analysis of the fluorescence intensity of the streptavidin (left) and hMSCs (right) before and after mixing. Neither the Alexa Fluor 700 streptavidin nor Ds-Red expressing hMSCs exhibited significantly different fluorescence in unmixed or mixed states.

FIGS. 5-7 are line graphs demonstrating the fluorescent intensity of each of the three molecular-weight variants of HA with (gray lines) or without (black lines) mixing with the hMSCs.

FIG. 8 provides IVIS images of mouse hindlimbs after a hyaluronic acid (HA)-200 injection.

FIG. 9 is a line graph demonstrating the fluorescent signal of an injection with streptavidin alone.

FIG. 10 is a line graph demonstrating the degradation patterns of HA of three molecular weights, as indicated by the fluorescent signals of Alexa Fluor 700.

FIG. 11 shows the coefficients a and b, obtained from an exponential regression analysis (to) (F(N)=ae^{-bx}).

FIG. 12 demonstrates the half-lives of HA-200, HA-800, and HA-1640.

FIG. 13 is a line graph demonstrating the hMSC fluorescent intensities in mixtures with each molecular-weight variant of HA.

FIG. 14 provides images of the immunofluorescence staining of the proliferation marker Ki-67. Representative images are shown for the three treatment groups, stained for Ds-Red and Ki-67 and with DAPI. Scale bar: 100 μm. Ki-67 and Ds-Red double-stained cells are indicated by arrows.

FIG. 15 is a bar graph demonstrating the percentage of Ki-67-positive cells among the Ki-67 and Ds-Red double-positive cells.

FIG. 16 provides images demonstrating results from a TUNEL assay double-stained with Ds-Red. Representative images are shown for each of the three treatment groups, stained for Ds-Red and apoptotic hMSCs and with DAPI. Scale bar: 100 μm. The TUNEL and Ds-Red double-stained cells are indicated by arrows.

FIG. 17 is a bar graph demonstrating the percentage of apoptotic hMSCs among the Ds-Red positive cells.

FIG. 18 to FIG. 20 demonstrate that a close relationship existed between hMSCs and different molecular weights of the HA at the early times.

FIG. 21 provides images of immunofluorescence staining of peri-injury hindlimb sections 3 days after the injection; in the color version of this figure, Red=Ds-Red, Green=tropomyosin, Blue=DAPI; scale bar: 100 μm.

FIG. 22 is a bar graph demonstrating the statistical analysis of the retained hMSC stained in FIG. 21.

FIG. 23 provides representative images of mouse ischemic hindlimbs after treatment.

FIG. 24 is a bar graph demonstrating that injections of HA-200, HA-800, or HA-1640 along with the hMSCs increased blood flow in the ischemic hindlimbs. The blood flow at days 0, 1, 7, 14, 21 and 28 in each experimental group was measured by laser Doppler flowmetry (*** P<0.001 vs. PBS-treated group; ### P<0.001, # P<0.05 vs. HA-800/h MSC group; +++P<0.001, ++P<0.01, +P<0.05 vs. HA-1640/h MSC group).

FIG. 25 is a line graph demonstrating the clinical scores of mice 7 to 28 days after the induction of hindlimb ischemia (### P<0.001 vs. PBS-treated, hMSC alone, HA-200 alone, HA-800 alone, HA-1640 alone, and HA-1640/hMSC groups; *** P<0.001, ** P<0.01 vs. HA-800/hMSC group).

FIG. 26 provides representative immunofluorescence images of isolectin demonstrating the capillary at the mid-thigh level for different treatment groups: sham (Group a), PBS (Group b), hMSCs (Group c), HA-200 (Group d), HA-800 (Group e), HA-1640 (Group f), HA-200/hMSC (Group g), HA-800/hMSC (Group h) and HA-1640/hMSC (Group i). The capillaries were labeled with anti-isolectin, the skeletal muscles were labeled with anti-tropomyosin, and the nuclei with stained with DAPI. Scale bar: 100 μm. The capillaries stained for isolectin and exhibiting circular shapes are indicated by arrows.

FIG. 27 is a bar graph demonstrating the quantification of the capillary density at the peri-injury region. *** P<0.001 vs. all other treatment groups.

FIG. 28 provides representative immunofluorescence images of smooth muscle 22a showing the arterioles at the mid-thigh level: sham (Group a), PBS (Group b), hMSCs (Group c), HA-200 (Group d), HA-800 (Group e), HA-1640 (Group f), HA-200/hMSC (Group g), HA-800/hMSC (Group h) and HA-1640/hMSC (Group i). The arterioles were labeled with anti-smooth muscle 22a, the skeletal muscles were labeled with anti-tropomyosin, and the nuclei with stained with DAPI. Scale bar: 100 μm. Arrows indicate arterioles stained against smooth muscle 22a.

FIG. 29 is a bar graph demonstrating the quantification of the arteriole density at the peri-injury region. *** P<0.001 vs. HA-800/hMSC group; ** P<0.01 vs. PBS-treated, hMSC alone, HA-200 alone, HA-800 alone, and HA-1640 alone groups.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless defined otherwise, all technical and scientific terms used herein have the same meaning as commonly understood by one of the ordinary skill in the art to which this invention belongs.

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

Here, the term “therapeutic cell” refers to cellular material to be introduced into and/or in the vicinity of a treatment site. Therapeutic cells vary with respect to characteristics such as formulation (including combination with a scaffold or other non-cellular component), the genetic relationship of the cells to the patient (autologous, allogeneic, xenogeneic), and the cell source. The in vivo biological activity and safety profile of the therapeutic cell are influenced by cell origin (donor source, tissue source), as well as the level of manipulation and stage of differentiation at the time of administration.

The term “cell population” as used herein refers to the number of therapeutic cells to be administered to the treatment site or the total number of therapeutic cells residing at the treatment site and cells proliferated from these residing therapeutic cells.

As used herein, the term “treatment site” is meant to refer to a desired site for administration of therapeutic cells and the biodegradable material of the present invention. “Treatment site” is thus meant to include, although is not necessarily limited to, a subcutaneous, intravenous, intrathecal, intraorbital, intraocular, intraaural, intratympanic, intramuscular, intra-arterial, intra-articular, intracavitary, intraductal, intraglandular, intravascular, intranasal, intraperitoneal, intraspinal, epidural, intracranial, intracardial, intrapericardial, peritumoral, or intratumoral (i.e., within a cancerous growth) site within a subject. “Treatment site” thus also encompasses intracavitary sites, e.g., sites within or near a selected organ or tissue (e.g., central nervous system (e.g., spinal fluid), kidney, liver, pancreas, heart (e.g., intrapericardial), lung, eye, inner ear, middle ear, cochlea, lymph nodes, breast, prostate, ovaries, testicles, thyroid, spleen, etc.), into arteries that feed a selected organ to tissue, or at a site associated with a microbial infection (e.g., bacterial, viral, parasitic or fungal infection).

Unless contrary to the context, the term “treatment” are used herein broadly to include a preventative (e.g., prophylactic), curative, or palliative measure that results in a desired pharmaceutical and/or physiological effect. Also, the terms “treatment” and “treating” as used herein refer to application of the present method to a subject in need of cell therapy, with the purpose to partially or completely alleviate, ameliorate, relieve, delay onset of, inhibit progression of, reduce severity of, and/or reduce incidence of one or more symptoms or features of a disease or condition. Generally, a “treatment” includes not just the improvement of symptoms or decrease of markers of the disease, but also a cessation or slowing of progress or worsening of a symptom that would be expected in absence of treatment.

The term “effective amount” as used herein refers to the quantity of a component (e.g., the biodegradable material) which is sufficient to yield a desired response (such as, maintaining the cell population and/or enhancing the therapeutic efficacy). Effective amount may be expressed, for example, in grams, milligrams or micrograms or as milligrams per kilogram of body weight (mg/kg). The term also refers to an amount of a pharmaceutical composition containing an active component or combination of components. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives.

As used herein, the term “therapeutically effective amount” refers to the quantity of an active component which is sufficient to yield a desired therapeutic response. A therapeutically effective amount is also one in which any toxic or detrimental effects of the compound or composition are outweighed by the therapeutically beneficial effects.

The term “subject” refers to a mammal including the human species that is treatable with the present method. The term “subject” is intended to refer to both the male and female gender unless one gender is specifically indicated.

As provided hereinbelow, the gross distribution of transplanted stem cells was determined through IVIS imaging and the results demonstrate that the degradation profiles of biodegradable materials affect the retention of transplanted cells. Accordingly, the present invention is based, at least, on the finding that biodegradable materials with specific degradation profiles are capable of augmenting cell retention and viability at the treatment site, thereby enhancing the therapeutic efficacy of cell therapies. Therefore, the present invention is directed to methods which apply the degradation characteristics of a biomaterial to optimize the retention of transplanted cells in vivo in order to maximize the therapeutic efficacy of the transplanted cells.

In one aspect, the present disclosure is directed to a method for maintaining a population of therapeutic cells administered to a treatment site in a subject for a period of time. Said method is particular useful for subjects in need of cell therapies; and hence, the present disclosure also contemplates a method for treating a subject in need of cell therapy.

According to certain embodiments, the method for maintaining a population of therapeutic cells administered to a treatment site in a subject for a period of time or the method for treating a subject in need of cell therapy comprises the steps of (a) administering a therapeutically-effective amount of therapeutic cells to the treatment site; and (b) administering an effective amount of a biodegradable material comprising a hyaluronan compound to the treatment site. The biodegradable material has an in vivo degradation profile similar to that of a hyaluronic acid having a molecular weight of 20 kDa to 2,000 kDa.

In various embodiments, the step (a) is performed before, at the same time with, or after the step (b). When steps (a) and (b) are performed concurrently administration, the therapeutic cells and the biodegradable material may be formulated as a single composition or in separate compositions.

In some embodiments of the present disclosure, the composition biodegradable material is up to about 5% by weight of the composition; such as 0.5, 1, 1.5, 2, 2.5, 3, 3.5, 4, 4.5, or 5% by weight. In some embodiments of the present disclosure, the amount of therapeutic cells is about 1×10⁴ to about 1×10⁸ cells in a single dosage volume of about 100 to about 2,000 μL. The composition according to the present disclosure may further comprise a pharmaceutically acceptable carrier or diluent.

As used herein the language “pharmaceutically acceptable carrier” is one that is suitable for use with the subjects without undue adverse side effects (such as toxicity, irritation, and allergic response) commensurate with a reasonable benefit/risk ratio. Also, each carrier must be “acceptable” in the sense of being compatible with the other ingredients of the pharmaceutical composition. Preferred but not exclusive carrier suitable for use in the present composition can be in the form of a solid, semi-solid, or liquid diluent. The composition may also comprise other additives such as growth factors, cytokines, chemokines, antitumor agents, antibacterial agents and antifungal agents, isotonic and absorption delaying agents and the like.

Also, steps (a) and/or (b) may be performed by directly applying, for example, during a surgery or for treatment sites locating at externally accessible area of the body, the therapeutic cells and/or the biodegradable material or compositions comprising the same to the treatment site, with or without an applicator (e.g., a dropper and the like). Alternatively, the direct injection also comprises injection of therapeutic cells and/or the biodegradable material or compositions comprising the same to the treatment site without the aid or assistance of other surgical or imaging devices. For example, the direct injection may comprise intradermal, subcutaneous, intramuscular, intravenous, intraosseous, intraperitoneal, intrathecal, epidural, intracardiac, intraarticular, intracavernous, or intravitreal injection. Still alternatively, the therapeutic cells and/or the biodegradable material or compositions comprising the same may be delivered with the aid of surgical or imaging devices. For example, the steps (a) and/or (b) may be performed during the open surgery, catheterization surgery, endoscopic surgery or robotic surgery. Imaging techniques suitable for use in the present method include, but are not limited to ultrasound, X-ray, computed tomography (CT), MRI, fluorescent and nuclear imaging (e.g., single-photon emission computed tomography (SPECT) and positron emission tomography (PET)). As could be appreciated, steps (a) and (b) may be carried out with the same or different administration route.

According to various embodiments of the present disclosure, the therapeutic cells suitable for use in the cell therapy may be stem cells, induced pluripotent cells, functionally differentiated cells or recombinant cells, or a combination thereof.

Tissue sources of stem cells include: adult (e.g., hematopoietic, vascular, neural, mesenchymal, cardiac, adipose, muscular, dental, skeletal, cartilage, periosteal, mammary, uterus, skin); perinatal (e.g., placental, umbilical cord blood); fetal (e.g., amniotic fluid, yolk sac, neural, skin); and embryonic. Stem cells or cell products derived therefrom are characterized by a variable capacity for self-renewing replication through cycles of cell division and the capacity for differentiation into a variety of cell types with specialized properties/functions. Such differentiation and replication are primarily controlled by the physiologic milieu of the host in which the cells reside following in vivo administration.

Functionally differentiated cells may be obtained from adult human donors (autologous or allogeneic) or from animal sources (xenogeneic). Source cells can include fibroblasts, chondrocytes, osteoblasts, osteocytes, adipocytes, epithelial cells, keratinocytes, retinal cells, dental cells, renal cells, pancreatic islet cells, hepatocytes, neuronal cells, immune cells, muscle cells, and blood cells. Functionally differentiated cells or cell products derived therefrom typically do not possess the property of self-renewing proliferation and the capacity to differentiate into multiple cell types; however, they may retain some cellular characteristics of their tissue of origin. Additionally, their characteristics may change after in vivo administration, based on specific extracellular cues.

Induced pluripotent cells (iPS cells) are cells that have been induced, either genetically or chemically, from differentiated somatic cells or stem/progenitor cells to cells having characteristics of higher potency cells, such as embryonic stem cells. iPS cells exhibit morphological, functional and growth properties similar to embryonic stem cells.

Recombinant cells are cells into which a recombinant gene has been introduced. For example, the therapeutic cells may be recombinant CD31+, CD34+, CD45+, CD133+, c-kit+, sca-1+ or isl-1+cells.

According to certain embodiments of the present disclosure, the hyaluronan compound may be hyaluronic acid or a derivative thereof. Hyaluronic acid (HA) is an anionic, non-sulfated glycosaminoglycan consisting of repeating disaccharide units of N-acetylglucosamine and D-glucuronic acid. Different numbers of the disaccharide subunit result in various molecular weights for HA, ranging from 20 kDa to 20,000 kDa. HA is an essential component of the extracellular matrix and considered to be an immuno-neutral polysaccharide; hence, it has been widely used in biomedical applications for decades.

Derivatives of hyaluronic acid include, but are not limited to, partial or total esters of hyaluronic acid, adipic dihydrazide-modified hyaluronan, amides of hyaluronan, crosslinked hyaluronic acid, hemiesters of succinic acid, heavy metal salts of hyaluronic acid, sulphated hyaluronic acid, N-sulphated hyaluronic acid, amine-modified hyaluronic acid, and diamine-modified hyaluronic acid. Hyaluronan compounds also include composites of hyaluronan and silk, hyaluronic acids cross-linked with other natural or synthetic materials. Derivatives or composites of hyaluronic acid can be obtained by chemically modifying one or more functional groups (e.g., carboxylic acid group, hydroxyl group, reducing end group, N-acetyl group) of hyaluronic acid and/or crosslinking hyaluronan with other molecules using methods known in the art.

According to various embodiments of the present disclosure, the biodegradable material consists of hyaluronic acid having a molecular weight of 20-2,000 kDa; preferably, 50-1,200 kDa; and more preferably, 200-800 kDa. For example, the molecular weight of the hyaluronan compound is 20, 30, 40, 50, 60, 70, 80, 90, 100, 110, 120, 130, 140, 150, 160, 170, 180, 190, 200, 200, 250, 300, 350, 400, 450, 500, 550, 600, 650, 700, 750, 800, 850, 900, 950, 1,000, 1,100, 1,200, 1,300, 1,400, 1,500, 1,600, 1,640, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900 or 5,000 kDa.

Alternatively, the biodegradable material may comprise two or more hyaluronan compounds. In certain embodiments, the biodegradable material may optionally comprise at least one biopolymer; examples of which include, but are not limited to, collagen, gelatin, alginate, chitosan, fibronectin, and fibrin glue. In these cases, the overall in vivo degradation profile of the biodegradable material should be similar to that of a hyaluronic acid having any of the above-identified molecular weights.

In certain embodiments of the present disclosure, the biodegradable material has an in vivo half-life of 4 hours to 28 days; alternatively, the in vivo half-life of the biodegradable material is 8 hours to 7 days, 12 hours to 5 days or 1-3 days after the administration. For example, the in vivo half-life may be 4, 5, 6, 7, 8, 9, 10, 11, 12, 13, 14, 15, 16, 17, 18, 19, 20, 21, 22, or 23 hours, or 1, 2, 3, 4, 5, 6, 7, 8, 9, 10, 11, 12, 13, 14, 15, 16, 17, 18, 19, 20, 21, 22, 23, 24, 25, 26, 27 or 28 days.

As could be appreciated, the therapeutic cells, once ministered to the therapeutic cells may be degraded; however, the experimental data provided hereinbelow demonstrates that the administration of the present biodegradable material may slow down the removal of therapeutic cells, thereby retaining a higher percentage of the therapeutic cells residing at the treatment site. Moreover, the experimental data also suggest that the biodegradable material may prevent cell apoptosis and promote cell proliferation in vivo. Accordingly, the administration of the present biodegradable material facilitates the maintenance of the cell population at the treatment site at least by retarding cell degradation and promoting cell proliferation. According to embodiments of the present disclosure, the population of therapeutic cells at the treatment site is maintained for at least 7 days; preferably at least 14 days; and more preferably, at least 28 days. Put it in another way, there are therapeutically-effective amount of therapeutic cells at the treatment sites at 1, 2, 3, 4, 5, 6, 7, 8, 9, 10, 11, 12, 13, 14, 15, 16, 17, 18, 19, 20, 21, 22, 23, 24, 25, 26, 27, or 28 days after the administration.

The present invention is also advantageous in that the method provides a greater amount of therapeutic cells during the early period (e.g., the first 10 days) of cell therapy. For example, the cell population at the treatment site may remain substantially the same or greater with respect to the initially administered therapeutic cells, at 3, 4, 5, 6, 7, 8, 9, or 10 days after the administration.

According to various embodiments, the therapeutically-effective amount of the therapeutic cells is 1×10⁴ to 1×10⁸ cells/kg body weight of a subject. According to certain embodiments, the effective amount of the hyaluronan compound is 0.01 to 10 mg/kg body weight of the subject. Preferably, the subject is an adult human weighted between 45-80 kg. Equivalent doses for children or other mammals could be estimated by conversions formulae known in the art; such as those provided in FDA's “Guidance for Industry: Estimating the Maximum Safe Starting Dose in Initial Clinical Trials for Therapeutics in Adult Healthy Volunteers.”

According to embodiments of the present disclosure, the present method is suitable for use in the treatment of various diseases or conditions treatable by cell therapy. For example, the subject may suffer from a damaged or infected tissue, a degenerative disease, or a cardiovascular disease such as coronary artery disease, cardiomyopathy, myocardial infarction, atherosclerosis, heart failure, a congenital heart disease, a peripheral artery occlusive disease, a valvular heart disease, Raynaud's phenomenon, Berger's disease and other connective tissue disorder associated vascular inflammation or damage, peripheral arterial disease, and an ischemic heart disease.

Additionally, the present method may be applicable in the treatment of diseases or conditions associated with bone, cartilage, muscle, eye, retina, nose, ear, thyroid gland, parathyroid gland, skin, muscle, bone, tooth, gingiva, wound, brain, spinal cord, breast, uterus, ovary, testis, liver, pancreas, or kidney. Moreover, the present method may be adapted to the field of cosmetic or reconstructive medicine.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

Materials and Methods

Hyaluronic Acid (HA).

Biotinylated hyaluronic acids of 200 kDa, HA 800 kDa and HA 1640 kDa were purchased from Creative PEGworks, Winston-Salem, N.C., USA and stored at 4° C.

Conjugating Fluorescently Labeled HA.

Biotinylated HA was suspended in deionized water overnight, and Alexa Fluor 700-conjugated streptavidin (Invitrogen, Grand Island, N.Y., USA) was prepared according to the manufacturer's instructions. The streptavidin and biotinylated HA were then mixed and gently agitated at 4° C. This mixture was filtered through Amicon centrifugal filter units (molecular-weight cut off 100 kDa; Millipore, Billerica, Mass., USA) to exclude extra streptavidin. The final products were freeze-dried and stored at 4° C.

Human Mesenchymal Stem Cells (hMSC) Culture.

hMSCs were cultured in minimum essential alpha medium (αMEM) containing 20% fetal bovine serum (FBS) and 4 ng/ml human fibroblast growth factor-basic (bFGF; Invitrogen, Grand Island, N.Y., USA). hMSCs were maintained at 37° C. and an atmosphere of 5% CO₂ in air on culture dishes. Cells were trypsinized and counted under light microscopy before transplantation.

Experimental Animals.

All animal research procedures were approved by the Experimental Animal Committee, Academia Sinica, Taipei, Taiwan, R.O.C. 8-week-old male nude mice from the National Laboratory Animal Center were used. All animals were anesthetized with Zoletile (50 mg/kg; Virbac, France) and Rompun (0.2 ml/kg; Bayer Healthcare, Germany) before surgery and in vivo measurements.

Quantifying In Vivo Material Degradation and Cell Retention Using Fluorescent Signals.

hMSCs were cultured on 10-cm culture dishes and maintained at 37° C. under an atmosphere of 5% CO₂ in air. A total of 4×10⁶ hMSCs were mixed with 200 μl of αMEM culture medium or 1% HA and then injected into the ischemic muscles of mice. Immediately and at various intervals after the injection, the mice were imaged using a Xenogen IVIS® Spectrum device and the corresponding filter sets. The fluorescent intensity was determined by calculating the number of photons within the manually drawn ROI, and the intensity was adjusted to exclude tissue autofluorescence.

Animal Model of Hindlimb Ischemia and Treatment.

The overall surgical mortality rate was 0%. The left femoral artery and iliac artery in mice were ligated and then cut to induce hindlimb ischemia. 200 μl of PBS, 4×10⁶ hMSC (suspended in culture medium), 1% solutions of the three molecular weight HAs, or 1% solutions of the three molecular weight HAs mixed with 4×10⁶ hMSC were injected intramuscularly. The injections were delivered into 4 sites at the ischemic site (50 μl for each site).

Blood Flow Measurement.

Microvascular blood flow was measured on a laser Doppler imager (Moor Instrument, UK). The blood flow before operation, 1 day afterwards, and every week for the following 4 weeks was recorded for both limbs. The data are presented as the blood flow ratio of the ischemia limb (left) to the normal limb (right).

Clinical Scoring of Mice after Hindlimb Ischemia.

Clinical scores were estimated from daily observation of mice activity and hindlimb condition and were categorized into 7 stages from 0 (normal), 1-3 (muscle atrophy), 4-5 (occurrence of gangrene) to 6 (limb amputation).

Immunohistochemistry and Immunofluorescence Staining.

The fixed distal calf and thigh muscles were deparaffinized, rehydrated and boiled in 10 mM sodium citrate (pH 6.0) for 10 minutes, followed by incubation with antibodies against RFP, SM22α (Abcam, Cambridge, Mass., USA), Ki-67 (BD Biosciences, San Jose, Calif., USA), tropomyosin (DHSB, Iowa city, Iowa, USA) and isolectin (Invitrogen, Grand Island, N.Y., USA) at 4° C. overnight, and then incubated with Alexa Fluor 488- or 568-conjugated secondary antibodies (Invitrogen, Grand Island, N.Y., USA). After staining with DAPI (Sigma-Aldrich, St. Louis, Mo., USA), sections were mounted and observed under a fluorescence microscope. The capillary and arteriole densities at the border zone were measured and images were taken from 8 randomly-selected ischemic sites (200× magnification) in each sample and quantification was performed by manually counting each section; the 8 values were averaged.

Statistical Analysis.

All data are presented as mean±standard deviation unless otherwise indicated (n=3 for in vitro spectrum analysis, n=6 for in vivo HA degradation, hMSC retention profiles, n=8 for capillary and arteriole density analysis). For multiple comparisons, analysis of variance (ANOVA) with Bonferroni adjustment was performed. A probability value of P<0.05 was considered to represent statistical significance.

### Example 1

### Characterization of HA and hMSCs

As provided herein, a platform to simultaneously quantify biomaterial degradation and stem-cell retention was designed to evaluate the impact of material degradation kinetics on stem-cell retention. To simultaneously track in vivo biomaterial degradation and stem-cell retention over time, Alexa Fluor 700 labeled hyaluronan and Discosoma sp. Red (Ds-Red)-expressing hMSCs were used along with a non-invasive imaging system (IVIS; PerkinElmer, Waltham, Mass., USA) to reduce animal consumption and experimental variance while still revealing material retained in vivo.

Examination of the relationship between the fluorescent intensity and the concentrations of Alexa Fluor 700-labeled streptavidin and Ds-Red expressing hMSC was first performed to establish the standard correlation between the fluorescent signal and the concentration of biotin-labeled HA or the number of hMSCs.

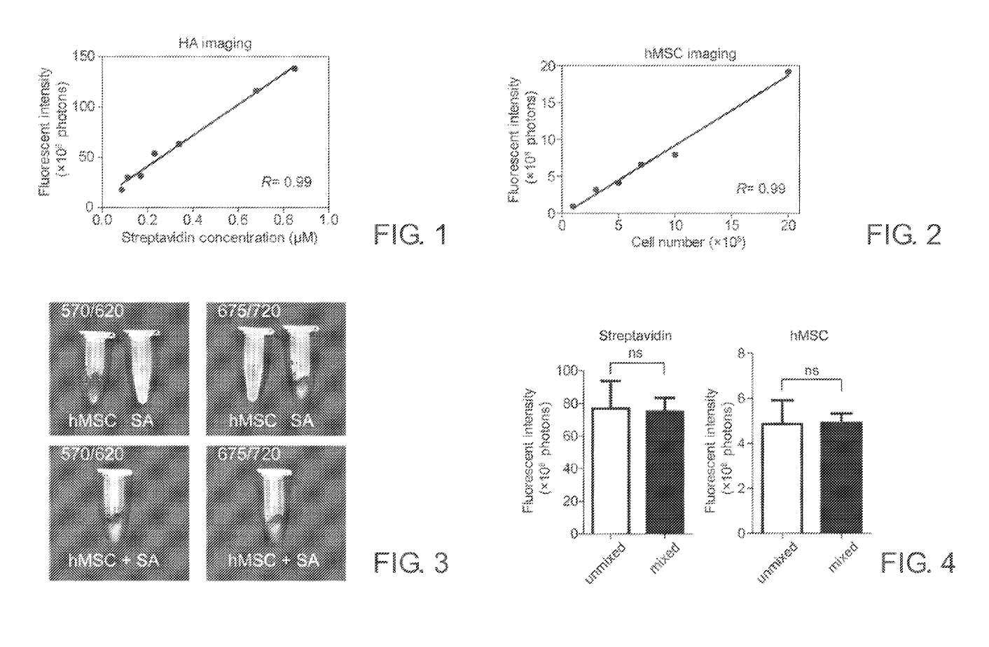

The fluorescent intensities of both HA and hMSCs increased linearly with the streptavidin concentration and hMSC counts, respectively (FIG. 1 and FIG. 2). Still referring to FIGS. 1 and 2, biotinylated HA labeled with streptavidin conjugated with Alexa Fluor 700 displayed higher fluorescence intensities than the Ds-Red expressing hMSCs.

### Example 2

### Fluorescence Spectrum Confirmation

The excitation and emission spectra of Alexa Fluor 700 and Ds-Red fall in the red and yellow-orange ranges, respectively. To ascertain whether these fluorescent signals could be adequately detected by IVIS and without causing spectral interference, Alexa Fluor 700 labeled streptavidin and Ds-Red expressing hMSCs were separately allocated in two microcentrifuge tubes (Eppendorf, Hamburg, Germany), and their original emission fluorescent signals were detected by the respective filter sets 675/720 (excitation/emission) for Alexa Fluor 700 and 570/620 for Ds-Red.

When using the filters for Ds-Red, signals from Alexa Fluor 700 were not detected, and the same was observed for the reverse situation (FIG. 3, upper two panels). Alexa Fluor 700 streptavidin and hMSCs were also mixed in a single tube, and the same two filter sets were again used to excite the sample. During this imaging process, the two filter sets were used to detect fluorescent signals emitted by either Alexa Fluor 700 or Ds-Red (FIG. 3, lower two panels).

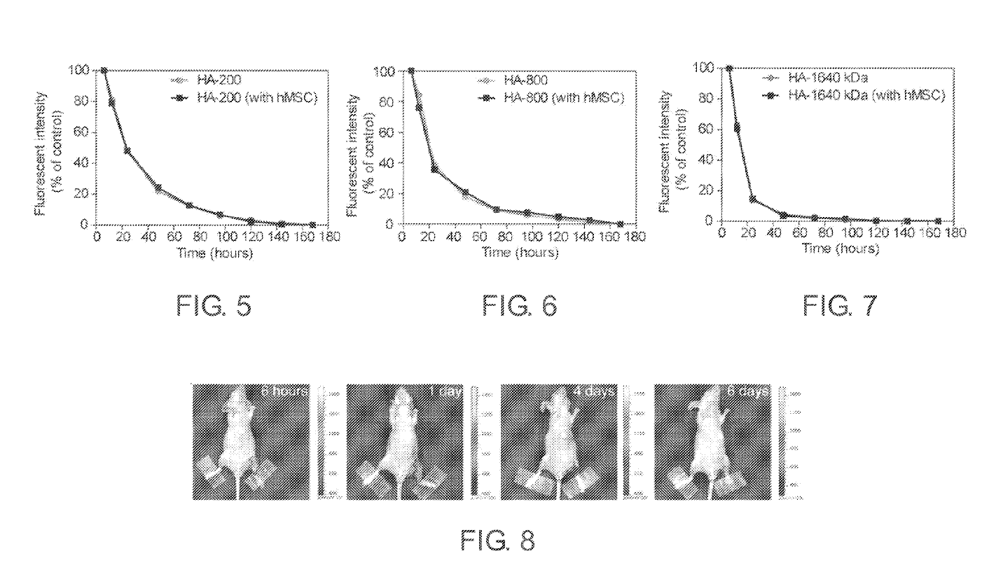

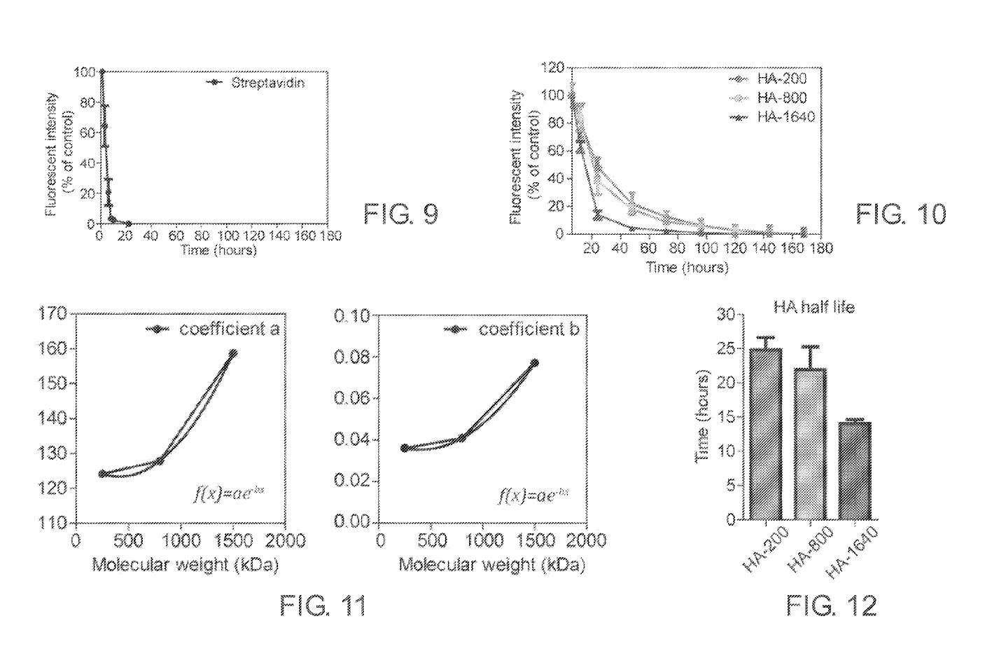

Regions of interest (ROIs) were drawn to evaluate the fluorescent intensities before and after mixing the streptavidin and hMSCs together. There was no significant difference in the signal emitted by the unmixed or mixed streptavidin and hMSC samples, indicating that the spectra of Alexa Fluor 700 and Ds-Red did not interfere with each other (FIG. 4). This concept was further confirmed by injecting HA or HA/hMSCs into a mouse hindlimb; the fluorescent signals adhered to the same HA degradation patterns with or without addition of hMSCs (FIGS. 5-7).

### Example 3

### Examination of HA Degradation Behavior In Vivo

A murine model of hindlimb ischemia was employed to investigate the effects of various cell-retention profiles on mitigating peripheral arterial disease. To image the material and cells' in vivo behavior, biotinylated hyaluronan with fluorescently labeled streptavidin and Ds-Red-expressing hMSCs were used.

Tracking of in vivo HA degradation in an ischemic disease model was performed by injecting 200 μl of 1% HA of three different molecular weights (200 kDa, 800 kDa and 1,640 kDa), denoted here as HA-200, HA-800 and HA-1640, respectively, into the hindlimbs of mice immediately after surgery, and measuring the in vivo fluorescent signals within the ROI. A decrease in the fluorescent signal over time indicated HA degradation (FIG. 8).

An examination of whether the residue of streptavidin alone emitted a signal was performed by injecting Alexa Fluor 700 labeled streptavidin alone into the hindlimb, which revealed that the fluorescence disappeared rapidly within 12 hours (FIG. 9).

All of the injected HA variants lost their fluorescent intensities over time exponentially (FIG. 10). Still referring to FIG. 10, the fluorescent intensity of the HA with the lowest molecular weight, HA-200, decreased the slowest, implying that this HA was degraded more slowly than the HA variants with higher molecular weights. Meanwhile, the fluorescent intensity of HA-1640 decayed the fastest, particularly during the first 24 hours after the injection. The finding that the HA with a low molecular weight degraded more slowly than the high molecular weight HAs may be due to the increased erosion surface of the latter HA.

To further examine the exponential degradation profiles of the three HA variants, the results summarized in FIG. 10 were fitted using classic exponential decay formula. This analysis (data not shown) confirmed the exponential degradation characteristics of all three HA variants. The two coefficients, a and b, were calculated using the fitting results, respectively. To inspect the relationship between each coefficient and each HA variant, the coefficients were plotted against molecular weight of the HA on line charts (FIG. 11). Coefficient a represents the initial amount of the material immediately following the injection, and it was found that coefficient a was greater than 100, and the number became larger as the molecular weight of the HA increased. This result may be due to swelling of HA. Coefficient b is inversely proportional to the half-life of the HA. Based on this coefficient, it is confirmed that the half-life of HA decreases along with increases in its molecular weight (FIG. 12). Taken together, these two molecular weight-coefficient graphs (FIG. 11) renders the prediction on in vivo degradation profile of other molecular-weight variants of HA possible.

### Example 4

### Relationship of hMSC Retention and HA Degradation

Mesenchymal stem cells (MSCs) have been specifically demonstrated to be not only pro-angiogenic but also tolerant to hypoxic or serum-free conditions, which are analogous to the conditions in an ischemic disease model. As provided herein, human MSCs (hMSCs) were immortalized and genetically modified to continuously express the Ds-Red fluorescent protein using a non-viral transfection method. The disease model used herein is murine hindlimb ischemia, representing peripheral arterial occlusive disease, which as many as 10 million people in the United States suffer from.

To determine the relationship between the hMSC retention and HA degradation, 4×10⁶ hMSCs were mixed with HA and then the mixture was administered to the ischemic hindlimbs of mice.

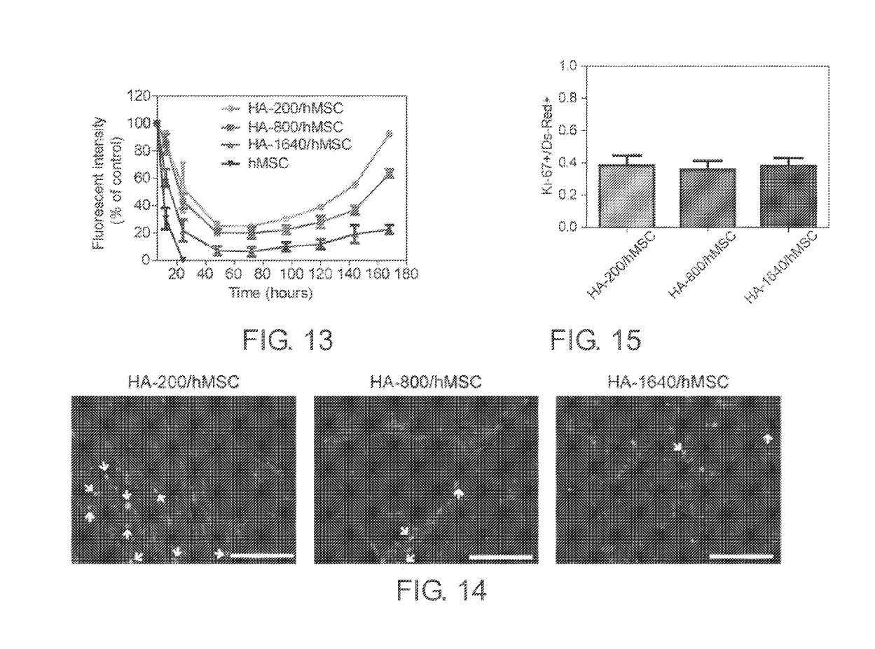

The fluorescent signals emitted from the hMSCs in the three combined treatment groups adhered to similar patterns (FIG. 13). In particular, during the first 48 hours after the injection, the signals decreased to 7% for HA-1640, 20% for HA-800, and 25% for HA-200 of the original intensity. This finding indicated a loss of the transplanted hMSCs, either into the circulation or due to hMSC apoptosis.

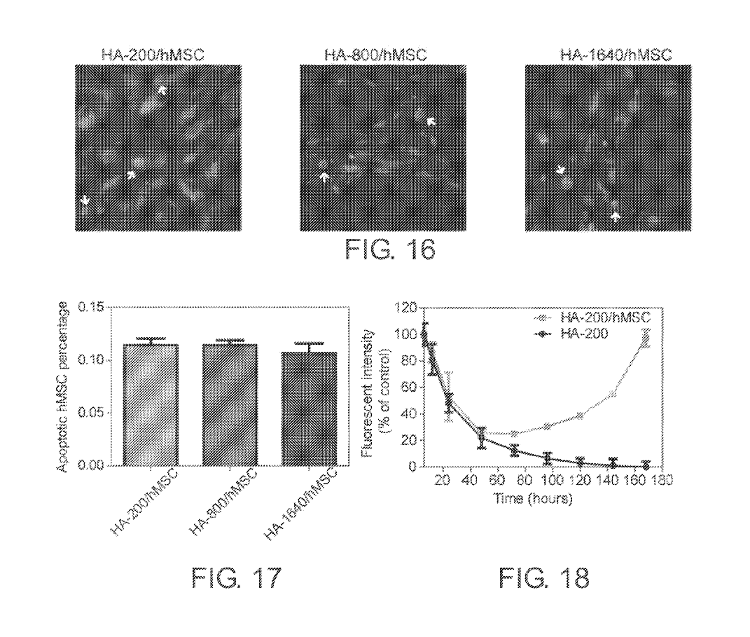

Still referring to FIG. 13, the decrease in the fluorescent signals from the hMSCs slowed and nearly reached a plateau 48 hours after the injection of HA/h MSCs, suggesting hMSC adhesion to the surrounding microenvironment. However, after 72 hours (or 3 days), the fluorescent signals started to increase, with the HA-200/hMSC group being the fastest to escalate, and the HA-1640/hMSC group being the slowest. This phenomenon was not caused by different cell proliferation rates induced by the different HA variants, for the percentage of proliferating hMSCs remained relatively the same among the treatment groups (FIGS. 14-15). Rather, the increase in fluorescence might be attributed to the proportion of hMSCs retained at 48 hours after the injection, as the HA-200/hMSC group retained approximately 3.6-fold more cells than the HA-1640/hMSC group, deciphering from the data in FIG. 13. The apoptotic level of the transplanted hMSCs was also examined, providing evidence that the fluorescence patterns of the combined therapy groups were not due to the different molecular weight of the HA variants (FIGS. 16-17).

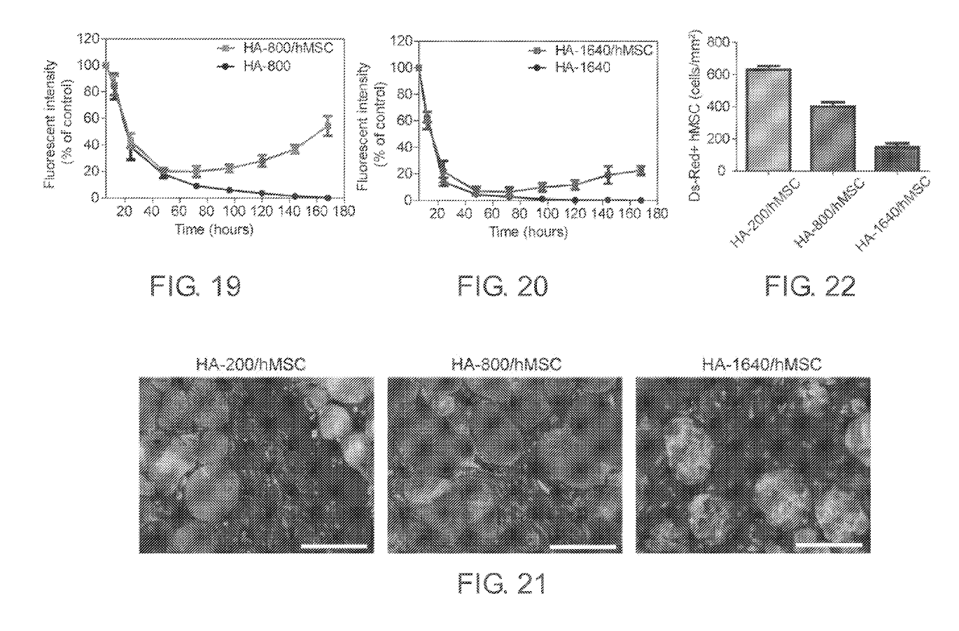

It is noted that the change of hMSC fluorescent intensity is similar to the corresponding HA degradation profiles in the first 48 hours after transplantation. After this time, the fluorescence of the hMSCs started to increase while the HA fluorescence continued to decrease (FIG. 18-20).

The peri-injury tissue sections obtained from the HA-200/hMSC, HA-800/hMSC, and HA-1640/hMSC treatment groups were stained for Ds-Red, which indicated the percentage of cells retained in each group. The highest numbers of hMSCs retained in the mouse hindlimbs were found in the HA-200 group, followed by HA-800 group (FIGS. 21 and 22).

From the data presented, the transplanted hMSCs likely underwent three stages: depletion, sedimentation, and proliferation. Within the first 48 hours after the injection, the hMSCs were depleted from the HA, as indicated by the decreasing trend of the fluorescent signal. Then, between 48 and 72 hours after transplantation, the hMSCs started to attach to the surrounding microenvironment, as indicated by stable fluorescence signals. The last stage occurred 72 hours after transplantation, when the cells began to proliferate, as evidenced by the elevated fluorescence signals even in the absence of HA. Accordingly, for successful delivery of cells, such as stem cells, the accompanying biomaterial should remain in the in vivo system for at least 48 hours. Otherwise, this material may not properly assist the cells in remaining at the site of injury.

### Example 5

### Therapeutic Efficacy of Different Treatment Groups

The progression of peripheral arterial occlusive disease can cause leg pain and severe morbidity, such as amputation. Alleviating the discomfort and preserving the limb at the same time are primary clinical goals for medication. As provided herein, the gross examination of the distal mouse thigh and calf muscles 1 day, 2 weeks, and 4 weeks after surgical excision of the left femoral artery allowed the direct observation of the level of limb salvage.

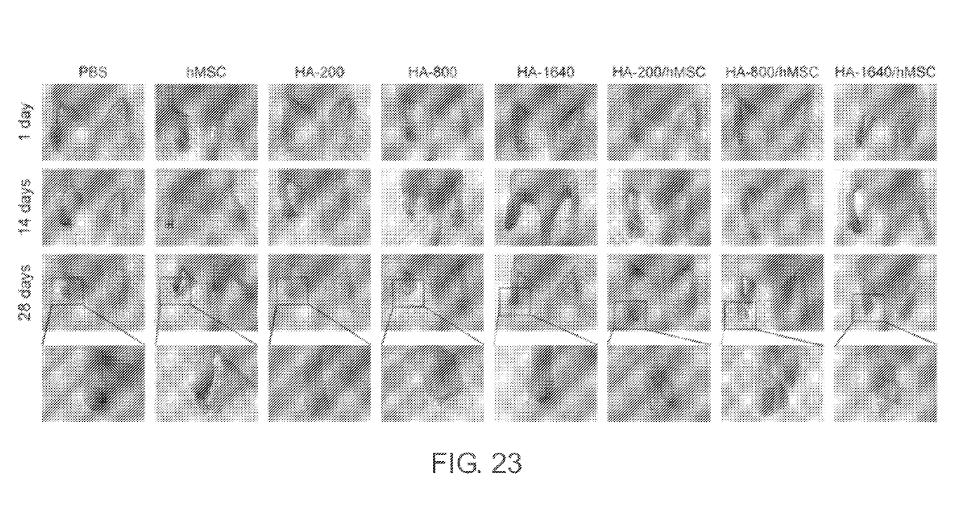

When only the hMSCs or HA was used to treat the ischemic hindlimbs, no obvious therapeutic benefit was observed, whereas the combined treatment with HA/hMSC significantly increased the preservation of the ischemic hindlimbs (FIG. 23). If a larger proportion of hMSCs was retained during the first 48 hours after administration, improved therapeutic results were observed, such as in the HA-200/hMSC group, which displayed successful limb salvage with only slight nail loss. However, if fewer hMSCs were retained, although limb amputation was not required, several toes were amputated (FIG. 23, rightmost column). For the other treatment groups, including hMSCs alone, HA-200 alone, HA-800 alone and HA-1640 alone, the ischemic hindlimbs exhibited no remarkable therapeutic outcomes compared to the PBS-treated control group, which experienced severe limb gangrene and necessary amputation.

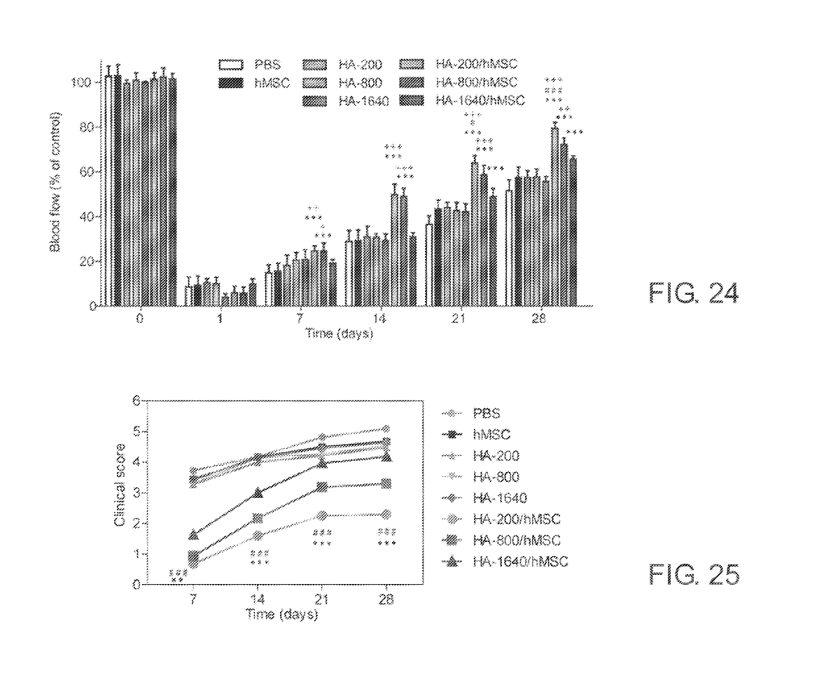

Another clinical index used to examine the therapeutic effects was the blood-flow recovery in the ischemic region. The blood flow in the mouse hindlimb was recorded by laser Doppler flowmetry (moorLDI; Moor Instrument, UK) every week after the injection to compare the recovery of blood flow between the different treatment groups (FIG. 24). Higher blood-flow recovery was found when animals were treated with HA-200 and hMSCs, whereas lower recovery was observed when HA-1640 was administered, in which fewer hMSCs were retained. More specifically, the blood flow in the HA-200/hMSC group elevated significantly beginning in the second week. In contrast, the groups receiving only hMSCs or HA alone exhibited no significant difference in blood-flow recovery in comparison to the PBS-treated group.

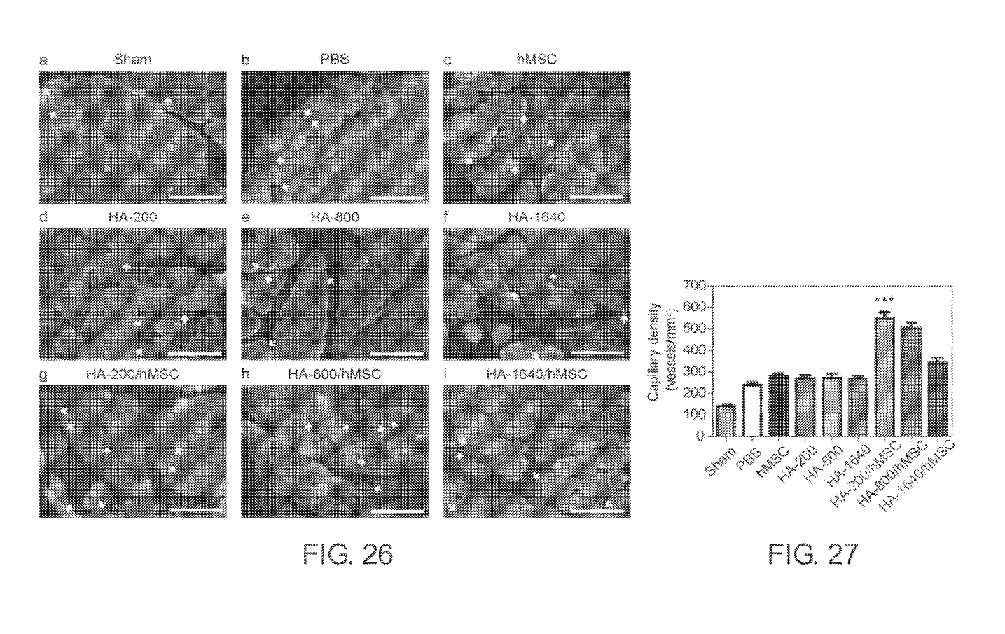

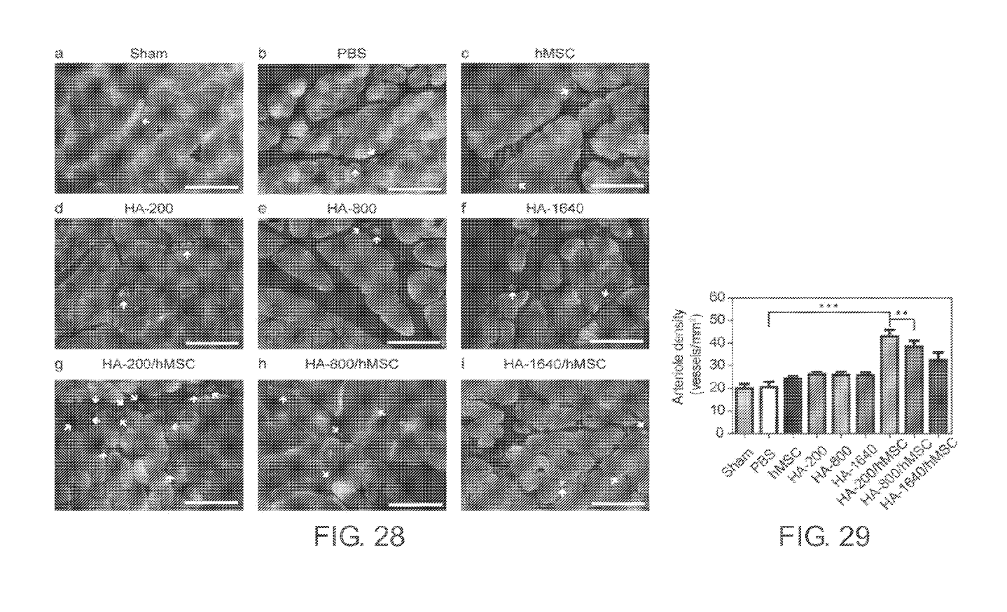

Clinical scores were calculated to further demonstrate the therapeutic outcomes of each treatment. Higher clinical scores were observed in the groups treated with PBS, hMSCs or HA alone, indicating the worsening condition of the ischemic hindlimbs, including serious limb amputations. In contrast, better therapeutic results were observed in the HA/hMSC combined therapy groups, with the HA-200/hMSC group yielded the best outcomes (FIG. 25). Furthermore, immunohistochemistry was performed to clarify the mechanism underlying improved therapeutic efficacy and found that through promoting angiogenesis (FIGS. 26 and 27) and arteriogenesis (FIGS. 28 and 29), the HA-200/hMSC treatment may facilitate the repair of the ischemic mouse hindlimb.

Through concurrent in vivo tracking of the HA degradation profiles and hMSC retention patterns, it was validated that enhanced stem-cell retention within a particular time window increases the therapeutic efficacy of the stem cells. Greater stem-cell retention in vivo rescued mice afflicted with hindlimb ischemia, increasing both limb preservation and blood-flow recovery by encouraging blood-vessel formation. Augmented stem-cell retention is dependent on using felicitous biodegradable materials whose degradation kinetics may vary with the injection site and injection volume. Optimized biomaterial degradation kinetics could be fine-tuned by material post-modification, molecular-weight selection or adjustment of the injection volume, which can only be accomplished by exploring the in vivo behavior of such biodegradable materials.

In sum, it was found that that the retention of transplanted stem cells is closely related to the remaining biomaterial. It was also found that therapeutic effectiveness is affected by stem cell retention. These results demonstrate that low-molecular-weight hyaluronan having a slow degradation and high cell-retention profile, improves the therapeutic efficacy of transplanted cells such as transplanted human stem cells. Further, the findings from Example 5 demonstrate that using biodegradable materials to facilitate stem-cell delivery has synergistic effects and that attention should be paid to the in vivo retention time when selecting the most appropriate biomaterial.

Although the experiments herein are material- and disease model-specific, the present invention can be adapted to other materials and cells or drug delivery systems. Moreover, the disease model could be extended to other ischemic diseases, such as myocardial infarction and stroke to improve the therapeutic outcomes of these ischemic diseases.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for maintaining a population of therapeutic cells administered to a treatment site in a subject in need of cell therapy for a period of time, comprising the steps of,
administering a therapeutically-effective amount of therapeutic cells to the treatment site; and
administering an effective amount of a biodegradable material comprising a hyaluronan compound to the treatment site, wherein the biodegradable material has an in vivo degradation profile similar to that of a hyaluronic acid having a molecular weight of 20 kDa to 2,000 kDa.

2. The method of claim 1, wherein the therapeutic cells are stem cells, induced pluripotent cells, functionally differentiated cells or recombinant cells.

3. The method of claim 2, wherein the stem cells are selected from the group consisting of, embryonic stem cells, hematopoietic stem cells, neural stem cells, vascular stem cells, mesenchymal stem cells, cardiac stem cells, adipose stem cells, muscular stem cells, dental stem cells, skeletal stem cells, cartilage stem cells, periosteal stem cells, mammary stem cells, uterus stem cells, endothelial stem cells, skin stem cells, placental stem cells, umbilical cord blood stem cells, yolk sac stem cells, and amniotic fluid stem cells.

4. The method of claim 2, wherein the functionally differentiated cells are selected from the group consisting of, fibroblasts, chondrocytes, osteoblasts, osteocytes, adipocytes, epithelial cells, keratinocytes, retinal cells, dental cells, renal cells, pancreatic islet cells, hepatocytes, neuronal cells, immune cells, muscle cells, and blood cells.

5. The method of claim 1, wherein the hyaluronan compound is at least one compound selected from the group consisting of, hyaluronic acid, partial or total esters of hyaluronic acid, adipic dihydrazide-modified hyaluronan, amides of hyaluronan, crosslinked hyaluronic acid, hemiesters of succinic acid, heavy metal salts of hyaluronic acid, sulphated hyaluronic acid, N-sulphated hyaluronic acid, amine-modified hyaluronic acid, diamine-modified hyaluronic acid, and a composite of hyaluronic acid and silk.

6. The method of claim 1, wherein the molecular weight of the hyaluronic acid is 50 kDa to 1,600 kDa.

7. The method of claim 6, wherein the molecular weight of the hyaluronic acid is 200 kDa to 800 kDa.

8. The method of claim 1, wherein the period of time is at least 7 days.

9. The method of claim 8, wherein the period of time is at least 28 days.

10. The method of claim 1, wherein the biodegradable material has an in vivo half-life of 4 hours to 28 days.

11. The method of claim 10, wherein the in vivo half-life is 8 hours to 7 days.

12. The method of claim 11, wherein the in vivo half-life is 1 to 3 days.

13. The method of claim 1, wherein the biodegradable material further comprises at least one component selected from the group consisting of, collagen, gelatin, alginate, chitosan, fibronectin, and fibrin glue.

14. The method of claim 1, wherein the population of the therapeutic cells at the treatment site, at 3-10 days after the administration, is substantially the same as or greater than the population of the therapeutic cells initially administered.

15. The method of claim 1, wherein the therapeutic cells are administered prior to or after the administration of the biodegradable material.

16. The method of claim 1, wherein the therapeutic cells are administered concurrently with the administration of the biodegradable material.

17. The method of claim 16, wherein the therapeutic cells and the biodegradable material are formulated as a single composition.

18. The method of claim 1, wherein the therapeutic cells and the biodegradable material are administered by direct application, catheter-assisted delivery, endoscope-assisted delivery, robotic-assisted delivery, device-assisted delivery, or imaging device-guided delivery, respectively or concomitantly.

19. The method of claim 1, wherein the therapeutically-effective amount of the therapeutic cells is 1×10⁴ to 1×10⁸ cells/kg body weight of the subject.

20. The method of claim 1, the effective amount of the hyaluronan compound is 0.01 to 10 mg/kg body weight of the subject.
